(19) United States
(12) Patent Application Publication
(10) Pub. No.: US 2023/0381037 A1
(43) Pub. Date: Nov. 30, 2023

(54) TIME DELAY PRODUCT DISPENSER

(71) Applicant: PERIOD PACK SWEDEN AB, 70369 Orebro (SE)

(72) Inventors: Amanda MATTSSON, Orebro (SE); Lova SVENSSON, Orebro (SE)

(73) Assignee: PERIOD PACK SWEDEN AB, 70369 Orebro (SE)

(21) Appl. No.: 18/026,399

(22) PCT Filed: Sep. 10, 2021
(86) PCT No.: PCT/SE2021/050867

§ 371 Date: Mar. 15, 2023

(30) Foreign Application Priority Data
Sep. 18, 2020 (SE) 2030294-9

(51) Int. Cl. A61F 13/551

(52) U.S. Cl. CPC A61F 13/55175

(57) ABSTRACT

A mechanical time delay product dispenser includes a cabinet arranged with at least one release tray and at least one pushbutton arranged to a push rod arranged to a pinion where the pinion is arranged to at least one rotary damper.

### BACKGROUND AND SUMMARY

The invention relates to a vending machine that dispense feminine products. Specifically this invention relates to push button, time delayed, mechanically operated dispenser for tampons.

There are coin drop mechanisms and coin-operated release machines in various forms in the prior art. These previous inventions vary in sophistication and efforts to prevent against theft, weather conditions, and durability. Numerous inventions within the prior art prevent fraud by having complex electronic or mechanical schemes to authenticate currency. Other less complex coin drop mechanisms or coin-operated release machines require the user to rotate a handle by twisting a rotatable lever.

Rotatable levers are specifically difficult to use for disabled people and different regulations stipulates maximum force required to use the device. As an example, the Americans with Disabilities Act (ADA) requires operable parts of fixed elements to not require the user to tightly grasp, pinch, or twisting of the wrist, or more than 5 pounds of force (lbf) to operate.

For release machines, dispensers, arranged to be non-coin operated it is furthermore important to design a dispenser that is non-electrical, cost effective to produce, reliable and easy to fill with tampons.

Patent document U.S. Pat. No. 10,192,386 B2 discloses an dispenser and when a pushbutton is pressed in towards the centre of the cabinet of the dispenser, a push rod moves causing teeth on a push rod to engage teeth on a pinion gear causing a first wing and a second wing to rotate and push a lowest tampon off a tampon rack and onto a bottom release tray while causing a vertical plunger rod and a plunger to displace downwards and cause said the plunger to be temporarily affixed to a right timing plate, with a length of time to reset the pushbutton to dispense another tampon is dependent on a suction release timing of the plunger before it is released.

Patent document U.S. Pat. No. 10,192,386 B2 does not disclose a dispenser that is simple in construction, cost effective to produce and reliable.

Further problems addressed by the invention will become clear from the following detailed description of the various embodiments.

The present invention relates, according to an aspect thereof, to an improved mechanical time delay product dispenser comprising a cabinet arranged with at least one release tray, the cabinet is also arranged with at least one pushbutton arranged to a push rod arranged to a pinion where the pinion is arranged to at least one rotary damper.

According to further aspects of the improved mechanical time delay product dispenser, provision is made as follows;
that the rotary damper is arranged on the same axis as the pinion.
that the rotary damper is arranged to the pinion by a coupling.
that the mechanical time delay product dispenser is arranged with a liftable product magazine.
that the mechanical time delay product dispenser is arranged with two pushbuttons, a left pushbutton and a right pushbutton.

The invention also relates, according to an aspect thereof, to a product dispenser arranged with at least one pushbutton and a product magazine arranged for dispensing products from the product magazine through at least one release tray where the product magazine is liftable arranged in the product dispenser.

According to further aspects of the improved product dispenser, provision is made as follows;
that the product dispenser is mounted to a wall and the product magazine is tiltable towards the wall to lock the product magazine in the lifted position to enable the product magazine to be manually filled with tampons.
that the product dispenser is arranged with a recess to allow the product magazine to be gripped and lifted.

The disclosed invention is unique because, according to an aspect thereof, it contains a pushbutton release coupled with a simple mechanical delay. Electronic delays are known in the prior art. However, these require batteries or electrical power to operate. The mechanical delay of an aspect of the present invention is unique because it prevents a user from emptying the machine of free products within a short period of time without the use of an electronic delay or complicated mechanical delay.

Another advantage of an aspect of the disclosed invention is that it provides a mechanically operated time delay coupled with a pushbutton release without requiring a user to twist or apply torque to any portion of the machine to obtain the dispensed feminine product.

Another advantage of an aspect of the disclosed invention is that the mechanical functioning components of the invention and the products to be dispensed are primarily found within the invention's cabinet interior. The external components of the invention such as the release tray and the moveable pushbuttons are found on the front of the invention making the dispenser easy to clean.

It is desirable to provide a predominantly mechanical device for dispensing feminine products that does not require the rotation of a handle.

It is also desirable to provide a simple mechanical device that dispenses feminine products for free with a time delay that is not electronic. This mechanical time delay, as opposed to an electronic time delay, allows the present invention to function without the need of batteries or power. This reduces the cost of installation, operation and maintenance.

### BRIEF DESCRIPTION OF FIGURES

Different embodiments of the invention are described in more detail below with reference to the attached figures, in which:

FIG. 1 shows a time delay product dispenser according to an embodiment of the invention.

FIG. 2 shows a time delay product dispenser when the pushbutton is pressed according to an embodiment of the invention.

FIG. 3 shows a time delay product dispenser with the pushbutton housing removed according to an embodiment of the invention.

FIG. 4 shows a time delay product dispenser when the pushbutton is pressed with the pushbutton housing removed according to an embodiment of the invention.

FIG. 5 shows a time delay product dispenser with the product magazine elevated according to an embodiment of the invention.

FIG. 6 shows a time delay product dispenser with the product magazine elevated in a side view according to an embodiment of the invention.

FIG. 7 shows a time delay product dispenser with the product magazine elevated and tilted according to an embodiment of the invention.

FIG. 8 shows a time delay product dispenser with the product magazine elevated and tilted in a side view according to an embodiment of the invention.

### DETAILED DESCRIPTION

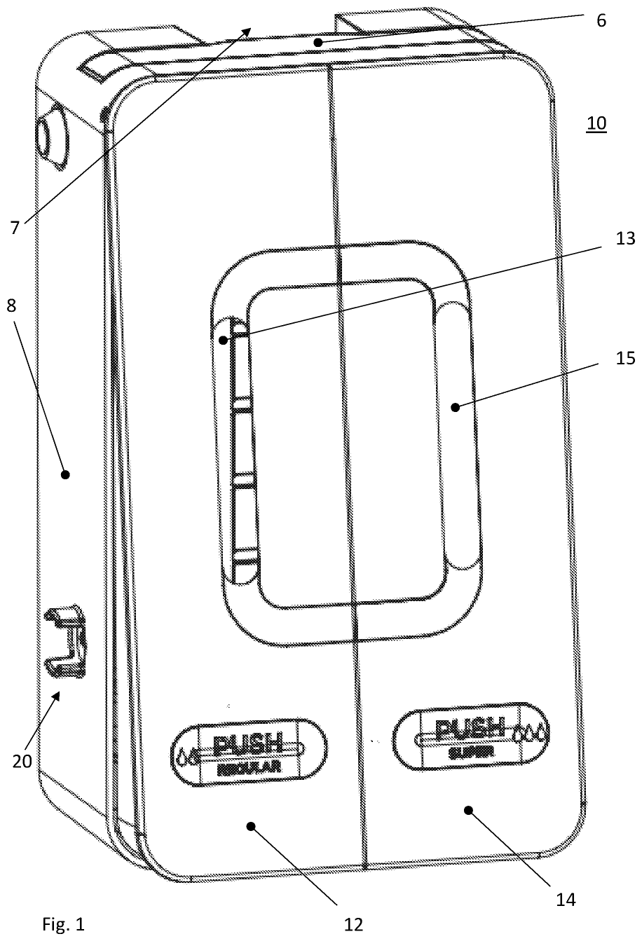

Referring to FIG. 1 there is illustrated a front perspective view of the present invention time delay product dispenser 10, also known as a product dispenser 10, arranged with a left pushbutton 12, also known as a first pushbutton 12, and a right pushbutton 14, also known as a second pushbutton 14. The present invention is comprised of internal components contained within the cabinet 8 of the time delay product dispenser 10. The left internal system will be discussed in the detailed description but there is a right internal system which functions independently but identically to the left internal system. In a standard configuration the left internal system allows a user to access tampons of regular size and the right internal system allows a user to access tampons of super-size. Cabinet 8 is comprised of a surrounding panel, a product magazine 6 and a back panel arranged to be fastened to a wall. A recess 7 is formed in the cabinet to allow the product magazine 6 to be gripped and lifted for filling the product magazine with new tampons. The cabinet 8 is arranged with a left release tray 20 and a right release tray (not shown in the figure). Located on the left pushbutton 12 is a left window 13. Similarly, located on the right pushbutton 14 is a right window 15. The windows serve to disclose if the product magazine is loaded with tampon. If the left internal system is empty the user of the time delay product dispenser 10 could use the right internal system to retrieve/eject a tampon and vice versa.

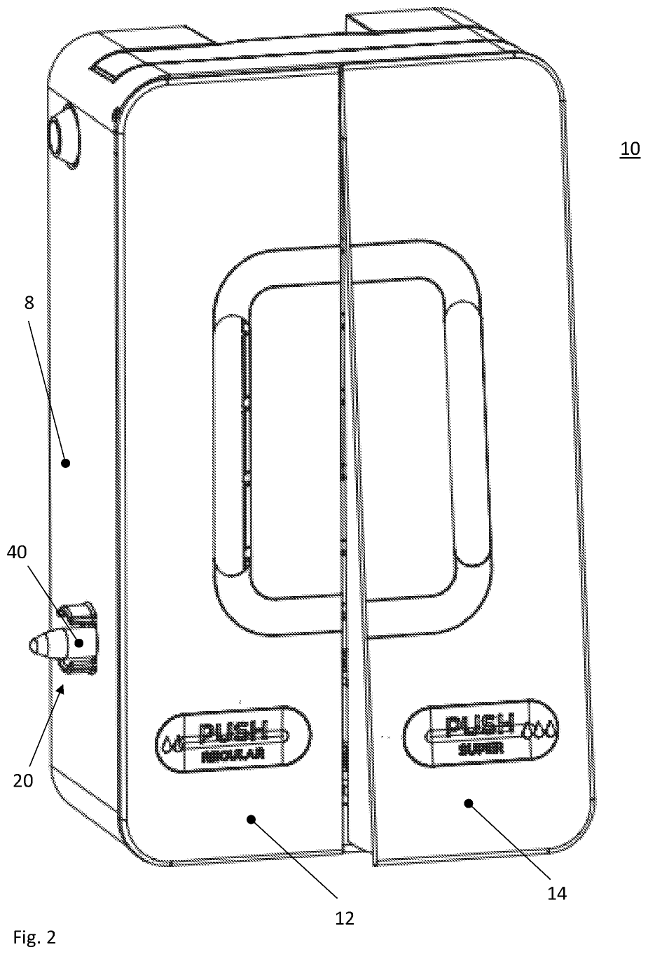

Referring to FIG. 2 there is illustrated a front perspective view of the present invention time delay product dispenser 10 arranged with a left pushbutton 12 and a right pushbutton 14 when the left pushbutton 12 is pressed. During operation a user has the ability to press in one of the pushbuttons 12, 14 to release a tampon. In FIG. 2 the left pushbutton 12 is pressed and a tampon 40 is ejected from the left release tray 20. After pressing the left pushbutton 12 the pushbutton is returned to it initial state, as shown in FIG. 1, with a time delay. Since there is a time delay between the possibility to press the pushbuttons 12, 14 the magazine of tampons could not be emptied by the user of the time delay product dispenser 10 in a short time and thus avoiding misuse of the dispenser.

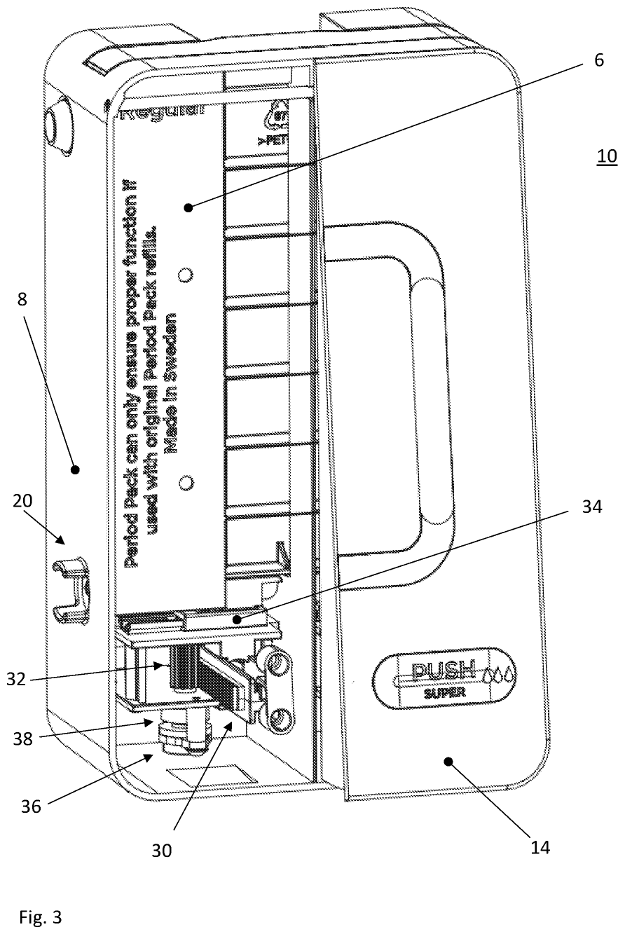

Referring to FIG. 3, there is illustrated the present invention the time delay product dispenser 10 in the open position with the pushbutton 12 console removed. Within the time delay product dispenser 10 is the product magazine 6 that hold the products to be dispensed, i.e. the tampons. When then push rod 30, arranged with teeth, moves inwards, this simultaneously causes the teeth of the push rod 30 that are interlocked with pinon gear teeth of the pinion gear 32 to rotate and causes the push compression spring, not shown in FIG. 3, to compress. The push compression spring is located between the push rod 30 and the back panel of the time delay product dispenser 10. Integrally affixed to the pinon gear 32 is the tampon pusher 34 that is in direct contact with lowest tampon 40 A rotary damper 36 is arranged in the axial extent of the pinion gear 32 providing a resistance when pushing the pushbutton 12 and a delay when the pushbutton 12 return to the initial state after dispensing a tampon. A rotary damper enables a mechanism to operate with a smooth, controlled motion. The damper is preferably filled with a viscous fluid, such as silicone. This fluid passes through rotating vanes, providing the damping resistance. The resistance force of a rotary damper is determined by the viscosity of the fluid and the design of the rotating vanes within the device. The rotary damper could be unidirectional, i.e. only dampening for a clockwise or counterclockwise motion, or bidirectional, i.e. dampening in both clockwise and counterclockwise direction. Commonly rotary dampers are arranged with a central axis, a rotary force could be arranged to the central axis of the rotary damper. The rotary damper 36 is arranged to the pinon gear 32 by a coupling 38. Commonly the rotary damper is arranged with a gear and the coupling 38 is arranged to fit the gear arranged on the rotary damper 36.

When the pushbutton 12 is pressed the rotary damper 36 and the push compression spring could provide a resistance during the pressing motion, if the rotary damper 36 is unidirectional only the compression spring could provide the resistance during the pressing motion. When the pushbutton 12 is released, the compression spring press the pushbutton 12 back to the initial condition. The push rod 30, that is arranged to the pushbutton 12, rotate the pinion gear 32 that is dampened by the rotary damper 36. When the pushbutton is fully depressed, the spring force of the push compression spring is high and thus move the pushbutton 12 back with greater force and speed compared to later stages in the process of returning the pushbutton to the initial state when the spring force of the compression spring is reduced. By selecting the dampening torque of the rotary damper and the spring constant of the compression spring the desired delay and force needed to press the pushbutton 12, 14 could be selected.

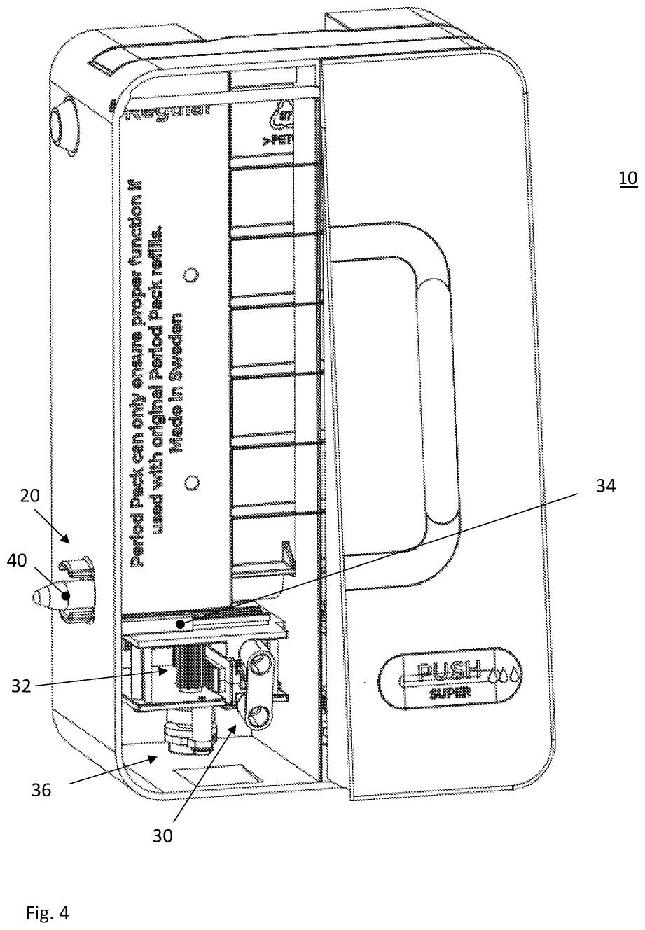

Referring to FIG. 4, there is illustrated the present invention the time delay product dispenser 10 in the open position with the pushbutton 12 console removed and the pushbutton pressed in towards the center of the time delay product dispenser 10 to a distance of approximately 2-5 cm, this causes the pinion gear 32 to rotate that also causes the tampon pusher 34 to push the lowest tampon 40 off the product magazine 6 out to the left release tray 20. When the lowest tampon 40 has been pushed off the product magazine 6 the second lowest tampon 41, not shown in FIG. 4, is pushed down by the weight of the remaining tampons in the product magazine 6 and thus becomes the lowest tampon 40. This process is repeated until all of the tampons are removed from time delay product dispenser 10. When all of the tampons are removed from time delay product dispenser 10 it is possible to view that the product magazine 6 is empty through the left window 13 and/or the right window 15.

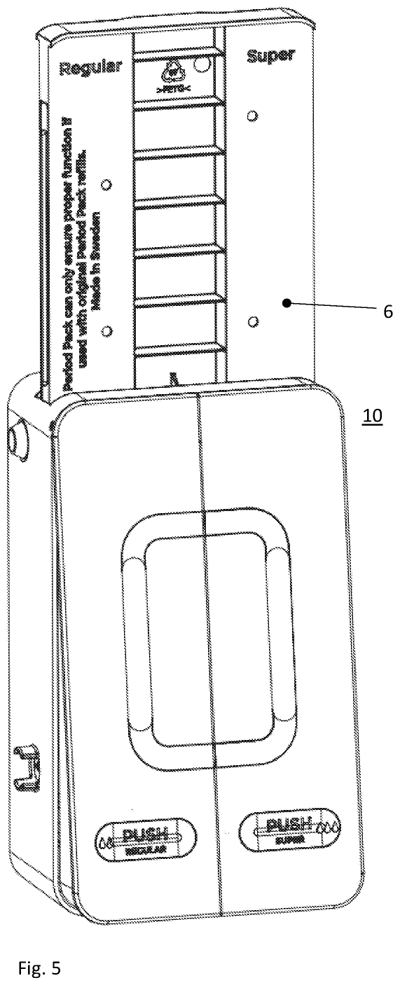

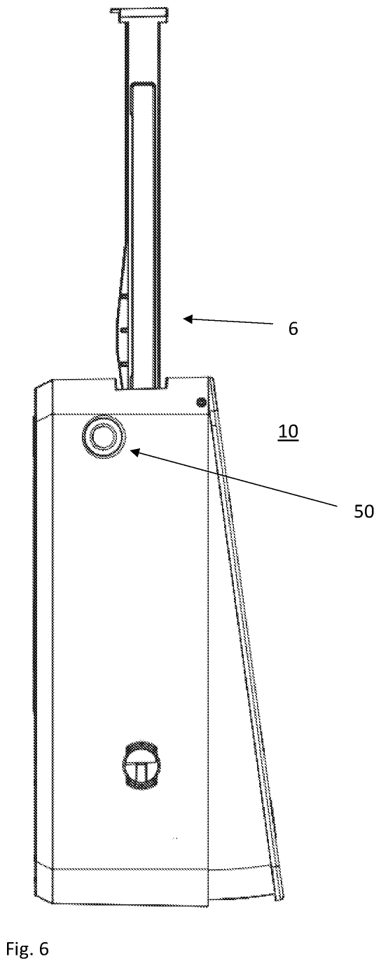

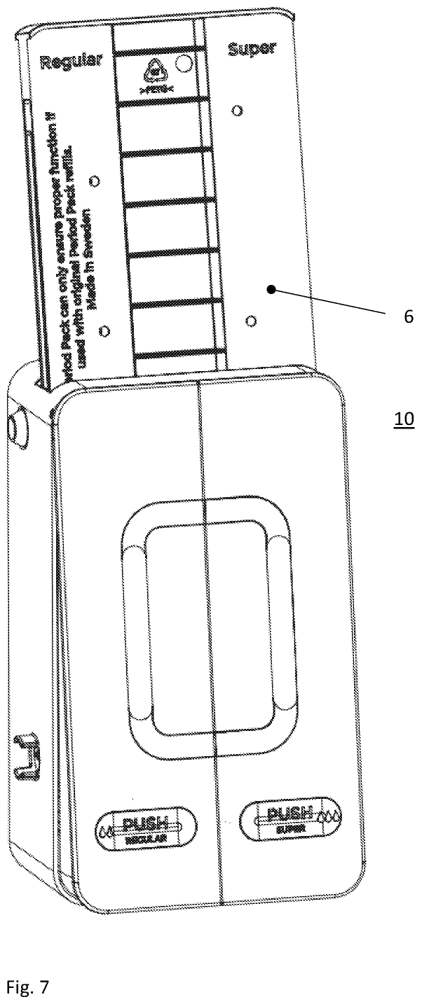

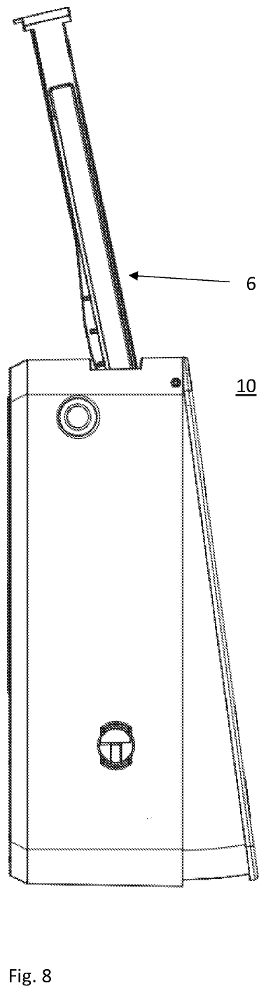

Referring to FIG. 5, FIG. 6, FIG. 7 and FIG. 8 there is illustrated the present invention the time delay product dispenser 10 when the product magazine 6 is lifted and/or drawn-out to enable filling of products, i.e. tampons, in the product magazine 6. In FIG. 5 the initial position when the product magazine 6 is lifted in the recess 7 is shown. In FIG. 6 the time delay product dispenser 10 viewed from the side is shown in the initial position when the product magazine 6 is lifted, a lock 50 is arranged that could be used to lock the product magazine 6 in the time delay product dispenser 10 to prohibit unauthorized personnel from removing the product magazine 6 from the time delay product dispenser 10. After lifting the product magazine 6 the magazine could be tilted/slanted toward the wall, as shown in FIG. 7 and FIG. 8. In this position it is possible to easy fill the product magazine 6 with new tampons manually. The product magazine 6 is arranged in the time delay product dispenser 10 so that it could be removed from the time delay product dispenser 10 when the time delay product dispenser is not mounted towards a wall. If the product magazine 6 is further tilted the product magazine 6 could be removed from the time delay product dispenser 10.

The invention is not limited to the particular embodiments shown but can be varied in different ways within the scope of the patent claims.

1-8. (canceled)

9. A mechanical time delay product dispenser, for tampons, comprising a cabinet arranged with at least one release tray and with at least one pushbutton arranged to a push rod arranged to a pinion and where a push compression spring is located between the push rod and a back panel of the time delay product dispenser, wherein the pinion is arranged to at least one rotary damper, and where a tampon pusher is integrally affixed to the pinion.

10. The mechanical time delay product dispenser according to claim 9, wherein the rotary damper is arranged on the same axis as the pinion.

11. The mechanical time delay product dispenser according to claim 10, wherein the mechanical time delay product dispenser is arranged with to two pushbuttons, a left pushbutton and a right pushbutton.

12. The mechanical time delay product dispenser according to claim 10, wherein the rotary damper is arranged to the pinion by a coupling.

13. The mechanical time delay product dispenser according to claim 12, wherein the mechanical time delay product dispenser is arranged with to two pushbuttons, a left pushbutton and a right pushbutton.

14. The mechanical time delay product dispenser according to claim 9, wherein the mechanical time delay product dispenser is arranged with to two pushbuttons, a left pushbutton and a right pushbutton.
